(19) United States
(12) Patent Application Publication
(10) Pub. No.: US 2023/0377048 A1
(43) Pub. Date: Nov. 23, 2023

(54) METHODS AND SYSTEMS FOR EVALUATING MEDICAL INSURANCE DATA IN SMART CITY BASED ON THE INTERNET OF THINGS

(71) Applicant: CHENGDU QINCHUAN IOT TECHNOLOGY CO., LTD., Chengdu (CN)

(72) Inventors: Zehua SHAO, Chengdu (CN); Xiaojun WEI, Chengdu (CN); Bin LIU, Chengdu (CN); Junyan ZHOU, Chengdu (CN); Yuefei WU, Chengdu (CN)

(73) Assignee: CHENGDU QINCHUAN IOT TECHNOLOGY CO., LTD., Chengdu (CN)

(21) Appl. No.: 17/810,621

(22) Filed: Jul. 4, 2022

(30) Foreign Application Priority Data
May 18, 2022 (CN) 202210538862.4

(51) Int. Cl. G06Q 40/08

(52) U.S. Cl. CPC G06Q 40/08

(57) ABSTRACT

Methods and systems for evaluating medical insurance data in a smart city based on the IoT are provided. The method includes: obtaining a risk query request of a user based on a service platform through a user platform, wherein the risk query request may be used to evaluate an insurance risk of an evaluation object; obtaining a related person of the evaluation object based on a population platform; obtaining medical features and insuring features of the evaluation object and the related person based on a medical information platform; determining a target medical feature and a target insuring feature based on the medical features and insuring features of the evaluation object and the related person; determining an evaluation value of the insurance risk based on the target medical feature and the target insuring feature; feeding back the evaluation value to the user through the user platform based on the service platform.

### CROSS-REFERENCE TO RELATED APPLICATIONS

This application claims priority to Chinese Patent Application No. 202210538862.4, filed on May 18, 2022, the contents of which are hereby incorporated by reference to its entirety.

### TECHNICAL FIELD

The present disclosure relates to the field of smart cities, and in particular, methods and systems for evaluating medical insurance data in a smart city based on the Internet of things (IoT).

### BACKGROUND

Nowadays, insurance companies and the types of business insurances are increasing, and different types of insurances of different insurance companies are reviewed at various modes. To control the risk, insurance companies need to perform a series of reviews on the insurance application information of customers before the customers apply for insurance.

Therefore, it is desirable to provide methods and systems for evaluating medical insurance data in a smart city based on the IoT, which may perform accurate and effective review on insurance customers.

### SUMMARY

One of the embodiments of the present disclosure provides a method for evaluating medical insurance data in a smart city based on the Internet of Things (IoT), which is performed by a medical management platform, including: obtaining a risk query request of a user based on a service platform and through a user platform, wherein the risk query request may be used to evaluate an insurance risk of the evaluation object; obtaining a related person of the evaluation object based on population information; obtaining medical features and insuring features of the evaluation object and the related person based on a medical information platform; determining a target medical feature and a target insuring feature based on the medical features and insuring features of the evaluation object and the related person; determining an evaluation value of the insurance risk based on the target medical feature and the target insuring feature; and feeding back the evaluation value to the user based on the service platform and through the user platform.

One of the embodiments of the present disclosure provides a system for evaluating medical insurance data in a smart city based on the Internet of Things, including a service platform and a medical management platform. The medical management platform may be configured to perform the following operations: obtaining a risk query request of a user based on a service platform and through a user platform, the risk query request may be used to evaluate an insurance risk of the evaluation object; obtaining a related person of the evaluation object based on a population information platform; obtaining medical features and insuring features of the evaluation object and the related person based on a medical information platform; determining a target medical feature and a target insuring feature based on the medical features and insuring features of the evaluation object and the related person; determining an evaluation value of the insurance risk based on the target medical feature and the target insuring feature; and feeding back the evaluation value to the user based on the service platform and through the user platform.

One of the embodiments of the present disclosure provides a computer-readable storage medium storing computer instructions, wherein when reading the computer instructions in the storage medium, a computer implements the method for evaluating medical insurance data in a smart city based on the IoT.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating the application scenario of a system for evaluating medical insurance data in a smart city based on the IoT according to some embodiments of the present disclosure;

FIG. 2 is an exemplary module diagram of the system for evaluating medical insurance data in a smart city based on the IoT according to some embodiments of the present disclosure;

FIG. 3 is an exemplary flowchart illustrating a method for evaluating medical insurance data in a smart city based on the IoT according to some embodiments of the present disclosure;

FIG. 4 is an exemplary schematic diagram illustrating the obtaining of a target medical feature according to some embodiments of the present disclosure;

FIG. 5 is an exemplary schematic diagram illustrating the obtaining of a target insuring feature according to some embodiments of the present disclosure;

FIG. 6 is an exemplary flowchart illustrating the determination of an evaluation value of an insurance risk based on a synergistic feature according to some embodiments of the present disclosure;

FIG. 7 is an exemplary schematic diagram illustrating the determination of an evaluation value of the insurance risk according to some embodiments of the present disclosure.

### DETAILED DESCRIPTION

To illustrate technical solutions of the embodiments of the present disclosure, a brief introduction regarding the drawings used to describe the embodiments is provided below. Obviously, the drawings described below are merely some examples or embodiments of the present disclosure. Those having ordinary skills in the art, without further creative efforts, may apply the present disclosure to other similar scenarios according to these drawings. It should be understood that the exemplary embodiments are provided merely for better comprehension and application of the present disclosure by those skilled in the art, and not intended to limit the scope of the present disclosure. Unless obvious according to the context or illustrated specifically, the same numeral in the drawings refers to the same structure or operation.

It should be understood that the terms “system”, “device”, “unit” and/or “module” used in the specification are means used to distinguish different components, elements, parts, segments or assemblies. However, these words may be replaced by other expressions if they serve the same purpose.

The terminology used herein is for the purpose of illustration only and is not intended to be limiting. As used herein, the singular forms “a,” “an,” and “the” may be intended to include the plural forms as well, unless the context clearly indicates otherwise; the plural forms may be intended to include the singular forms as well, unless the context clearly indicates otherwise It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

The flowcharts used in the present disclosure illustrate operations that systems implement according to some embodiments in the present disclosure. It is to be expressly understood, the operations of the flowchart may be implemented not in order. Conversely, the operations may be implemented in inverted order, or simultaneously. Moreover, one or more other operations may be added into the flowcharts. One or more operations may be removed from the flowcharts.

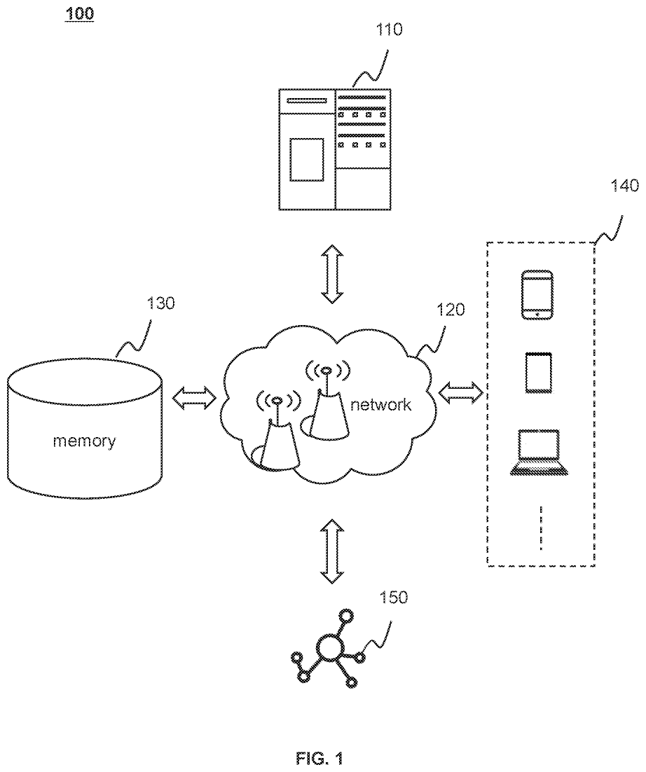

FIG. 1 is a schematic diagram illustrating the application scenario of a system for evaluating medical insurance data in a smart city based on the IoT according to some embodiments of the present disclosure.

As shown in FIG. 1, an application scenario 100 of a system for evaluating medical insurance data in a smart city based on the IoT may include a processor 110, a network 120, a memory 130, a terminal 140, and a network cloud platform 150. In some embodiments, components in the application scenario 100 may be connected (e.g., wireless connection, wired connection or the combination thereof) and/or communicate with each other through the network 120. For example, the processor 110 may be connected to the memory 130 through the network 120.

The system for evaluating medical insurance data in a smart city based on the IoT may determine an evaluation value of an insurance risk (the risk of participating in the insurance) of an evaluation object through implementing the method and/or process disclosed in the present disclosure. Specifically, when the evaluation object is willing to get insured, the insurance company may issue a risk query request to the system for evaluating medical insurance data in a smart city based on the IoT. In response to the risk query request, the system may determine the evaluation value of the insurance risk of the evaluation object based on medical features and insuring features of the evaluation object and the related person, so that insurance company may perform subsequent processing based on the evaluation value. For example, when the evaluation value of the insurance risk of the evaluation object is high, the insurance company may increase premium or even reject the request.

The processor 110 may process data and/or information related to the system for evaluating medical insurance data in a smart city based on the IoT. In some embodiments, the processor 110 may access to the information and/or data in the network cloud platform 150, the memory 130 and/or the terminal 140. For example, the processor 110 may obtain the medical features, insuring features, and synergistic features of the evaluation object and the related person from the memory 130 and/or the network cloud platform 150. In some embodiments, the processor 110 may process the information and/or data obtained from the network cloud platform 150 and/or memory 130. For example, the processor 110 may perform processing on the medical features and insuring features of the evaluation object and the related person to determine the evaluation value of the insurance risk of the evaluation object. In some embodiments, the processor 110 may include one or more processing engines (e.g., a single-chip processing engine or multi-chip processing engine). Just as an example, the processor 110 may include a central processing unit (CPU). The processor 110 may process the data, information and/or processing results obtained from other devices or system components, and perform program instructions based on these data to perform one or more functions described in the present disclosure.

The network 120 may include any suitable network that could promote the information and/or data exchange among the various components in the application scenario 100 of the system for evaluating medical insurance data in a smart city based on the IoT. The information and/or data may be exchanged through the network 120 among one or more components (e.g., the processor 110, the memory 130, the terminal 140, the network cloud platform 150) of the application scenario 100 of the system for evaluating medical insurance data in a smart city based on the IoT. For example, the network 120 may send the medical features, the insuring features and the synergistic features of the evaluation object and the related person obtained from the network cloud platform 150 to the processor 110. In some embodiments, the network 120 may be any one or both of the wired network and the wireless network. In some embodiments, the network 120 may include one or more network access points. For example, network 120 may include a wired or a wireless network access point. In some embodiments, the network may be point-to-point, shared, central, and of other topologies or of a combination of a plurality of topologies.

The memory 130 may be configured to store the data, instructions and/or any other information. In some embodiments, the memory 130 may store the data and/or information obtained from the processor 110, the network cloud platform 150, etc. For example, the memory 130 may store the medical features, insuring features and synergistic features of the evaluation object and the related person, as well as medical knowledge maps. In some embodiments, the memory 130 may be set in the processor 110. In some embodiments, the memory 130 may include a mass memory, a removable memory, etc. or any combination thereof.

The terminal 140 may refer to one or more terminal devices or software used by the user. In some embodiments, the terminal 140 may be mobile devices, tablet computers, laptops, etc. or any combination thereof. In some embodiments, the terminal 140 may interact with other components in the system for evaluating medical insurance data in a smart city based on the IoT through the network 120. In some embodiments, the terminal 140 can be a terminal device or software used by insurance personnel.

The network cloud platform 150 may be a cloud calculation platform communicatively connected with the system for evaluating medical insurance data in a smart city based on the IoT for storing and processing data. In some embodiments, the network cloud platform 150 may include a population information platform, a medical information platform, and a synergistic platform. The population information platform may refer to a cloud platform or an external database that records the information related to residents (such as ID information, address information of the residents, etc.). In some embodiments, the processor 110 may determine the related person of the evaluation object based on the population information platform. The medical information platform may refer to a cloud platform or an external database that records the related medical information of the residents (e.g., residents' disease records, medical treatment records, etc.).

In some embodiments, the processor 110 may obtain the medical features and insuring features of the evaluation object and the related person through the medical information platform. The synergistic platform may refer to one or more cloud platforms or external databases recorded with resident-related synergistic information. In some embodiments, the processor 110 may obtain the synergistic features of the evaluation object and the related person through the synergistic platform. In some embodiments, the network cloud platform 150 may be connect to the processor 110, the memory 130 and the terminal 140 for exchanging data through the network 120. For example, the network cloud platform 150 may send the obtained medical features and insuring features of the evaluation object and the related person to the processor 110 for processing.

It should be noted that the application scenario 100 of the system for evaluating medical insurance data in a smart city based on the IoT is only provided for the purpose of explanation, and is not intended to limit the scope of the present disclosure. For those skilled in the art, a variety of modifications or changes may be made according to the descriptions of the present disclosure. For example, the application scenarios may further include databases. For another example, the application scenario 100 may achieve similar or different functions on other devices. However, changes and modifications should not deviate from the scope of the present disclosure.

The IoT system is an information processing system that includes parts or all platforms of management platforms, service platforms, and user platforms. The management platform may realize the overall planning and coordination of the connection and cooperation between the functional platforms. The management platform brings together information about the IoT operation system, which may provide perceptual management and controlling management functions for the IoT operation system. The service platform refers to a platform providing input and output services for the user. The user platform refers to a functional platform performing perceptual information generation and control information execution.

The processing of information in the IoT system may be divided into processing flow of perceptual information and processing flow of control information. The control information may be information generated based on perceptual information. The processing of perceptual information may be that the user platform obtains the perception information and transmits it to the management platform. The control information may be issued by the management platform to the user platform to control the corresponding user.

In some embodiments, when applying the IoT system to urban management, it may be called as the smart city IoT system.

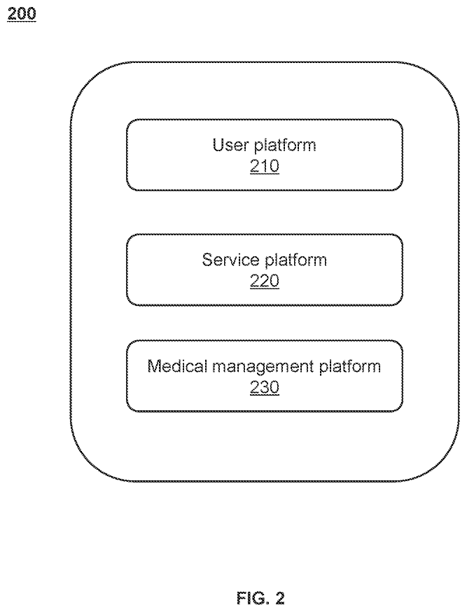

FIG. 2 is an exemplary module diagram of the system for evaluating medical insurance data in a smart city based on the IoT according to some embodiments of the present disclosure. As shown in FIG. 2, system 200 for evaluating medical insurance data in a smart city based on the IoT includes a user platform 210, a service platform 220, and a medical management platform 230. In some embodiments, the system 200 for evaluating medical insurance data in a smart city based on the IoT may be realized by part of the processor 110 or by the processor 110.

The user platform 210 may be a platform for interacting with users. In some embodiments, the user platform may be configured as a terminal device. For example, the terminal device may include mobile devices, tablet computers, or any combination thereof. In some embodiments, the user platform may be configured to receive requests and/or instructions input by the user. For example, the user platform may obtain the user's risk query request through the terminal (e.g., terminal 140). In some embodiments, the user platform 210 may be communicatively connected to (that is, interacting with) the service platform 220, send the input instructions to the medical management platform 230 via the service platform 220, and receive the data and/or information required by the user platform 210 via the service platform 220. The data and/or information may be extracted from the medical management platform 230.

The service platform 220 may be a platform for receiving and transmitting data and/or information. For example, the service platform 220 may obtain risk query requests from the user platform 210. For another example, the service platform 220 may feedback the evaluation value of the evaluation object to the user through the user platform 210.

The medical management platform 230 may refer to the overall planning and coordination of the connection and cooperation between the functional platforms, and gathers all information of system 200 for evaluating medical insurance data in a smart city based on the IoT. The medical management platform 230 may be a platform that provides perceptual management and controlling management functions for the system 200 for evaluating medical insurance data in a smart city based on the IoT. For example, the medical management platform 230 may obtain the user's risk query request through the service platform 210 and based on the user platform 210. The risk query request may be used to evaluate the insurance risk of the evaluation object. The medical management platform 230 may obtain the related person of the evaluation object based on a population information platform. The medical management platform 230 may obtain the medical features and the insuring features of the related person and the evaluation object based on the medical information platform, determine a target medical feature and a target insuring feature based on the medical features and insuring features of the evaluation object and the related person; determine an evaluation value of the insurance risk based on the target medical feature and the target insuring feature. Further, the medical management platform 230 may feedback the evaluation value based on the service platform 220 and through the user platform 210. The medical management platform 230 may include the processor 110 in FIG. 1 and other components. In some embodiments, the medical management platform 230 may be a remote platform controlled by managers, artificial intelligences, or preset rules.

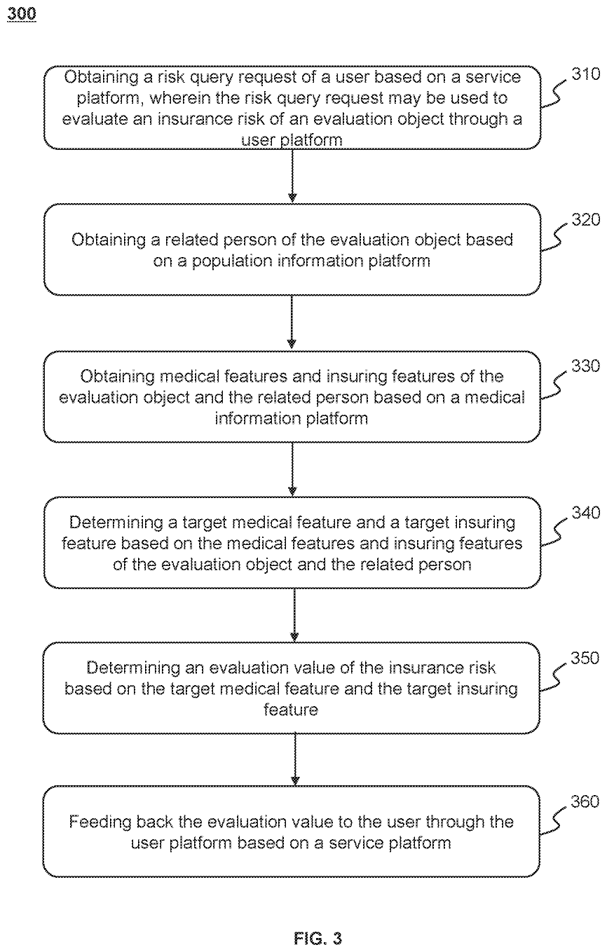

In some embodiments, the medical management platform 230 may further communicate with the network cloud platform 150 (e.g., the population information platform, the medical information platform, a synergistic platform, etc.) to obtain data and/or information. More about the population information platform, the medical information platform, and the synergistic platform, may be found in the relevant contents of FIG. 3-FIG. 5.

In some embodiments, the system 200 for evaluating medical insurance data in a smart city based on the IoT may be applied to various scenarios of medical insurance data evaluation, for example, the scenario of a new user insurance, a old user insurance renewal, etc. In some embodiments, the system 200 for evaluating medical insurance data in a smart city based on the IoT may obtain the medical features and the insuring features of the evaluation object and the related person in various scenarios, determine the evaluation value of the insurance risk of the evaluation object, and further determine the insurance strategy of the evaluation object.

A variety of scenarios of the system 200 for evaluating medical insurance data in a smart city based on the IoT may include the scenario of new user insurance and the old user renewal. It should be noted that the above scenarios are only for the purpose of illustration, and it does not limit the specific application scenario of the system 200 for evaluating medical insurance data in a smart city based on the IoT. Those skilled in the art may apply the system in other appropriate scenarios based on the content of the present disclosure.

Exemplarily, in the scenario of insurance for a new user, the related personnel of the insurance company may evaluate the insurance risk of the new user (that is, the evaluation object), and determine the insurance strategy of the new user based on the evaluation value. In the process of evaluating the insurance risk of new user, a related person of the new user may be obtained, and the medical features and the insuring features of the new user and the related person may be determined according to their medical data (the ages, the number of times of diseases, the treatment costs, etc.) and insuring data (for example, the historical insurance data), and determine the evaluation value of the insurance risk of the evaluation object (that is, the new user) based on the medical features and insuring features. The evaluation value of the insurance risk may be used to determine the risk for the insurance company to pay compensation due to an accident happened to the evaluation object after the evaluation object is insured. If the risk for the compensation is high, the insurance company may increase the premium or decline the new user's insurance request.

Exemplarily, in the scenario of the old user insurance renewal, the related personnel of the insurance company may evaluate the insurance renewal risk of the old user (that is, the evaluation object), and determine the renewal strategy of the old user according to the evaluation value. In the process of evaluating the risk of insurance renewal of the old user, the relevant data of the old user's historical insurance in the insurance company may be obtained, and further, the company may obtain the medical data and insuring data of the old user and the related person during the historical insurance period to determine the insurance renewal risk of the old user.

In some embodiments, the system 200 for evaluating medical insurance data in a smart city based on the IoT may include a plurality of medical insurance data evaluation sub-systems, and each sub-system may be applied to one scenario. The system 200 for evaluating medical insurance data in a smart city based on the IoT may comprehensively manage and process data obtained and output by each sub-system, and further obtain relevant strategies or instructions for assisting medical insurance data evaluation. For example, the system 200 for evaluating medical insurance data in a smart city based on the IoT may include a sub-system applied to the scenario of new user insurance, and a sub-system applied to the scenario of old user insurance renewal. The system 200 for evaluating medical insurance data in a smart city based on the IoT may be the superior system of each sub-system.

For those skilled in the art, after understanding the principles of the system, the system may be transferred to any other appropriate scenario without departing from this principle.

In some embodiments, the medical management platform 230 may be configured to: obtain a risk query request of a user through the service platform and based on the user platform, wherein the risk query request may be used to evaluate an insurance risk of the evaluation object; obtain the related person of the evaluation object based on the population information platform; obtain the medical features and the insurance features of the evaluation object and the related person based on the medical information platform; determine a target medical feature and a target insuring feature based on the medical features and insuring features of the evaluation object and the related person; determine an evaluation value of the insurance risk based on the target medical feature and the target insuring feature; and the medical management platform may feedback the evaluation value based on the service platform and through the user platform.

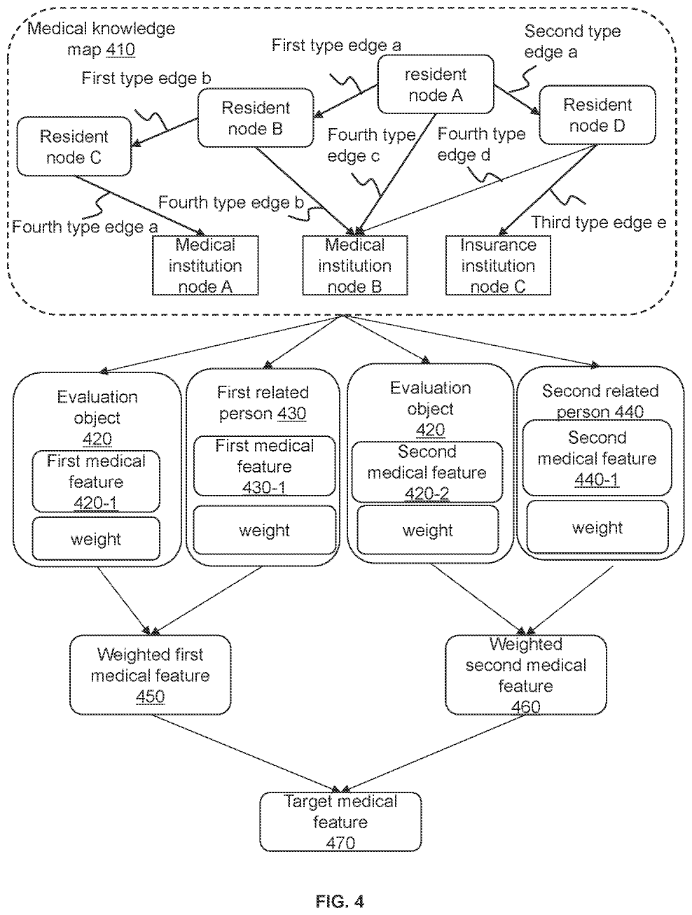

In some embodiments, the medical management platform 230 may be configured to further perform the following operations: obtaining a medical knowledge map based on the medical information platform; obtaining the medical features and insuring features of the evaluation object and the related person based on the medical knowledge map. For more details of the medical knowledge map, the medical features and the insuring features of the evaluation object and the related person, please refer to FIG. 3 and FIG. 4 and related descriptions.

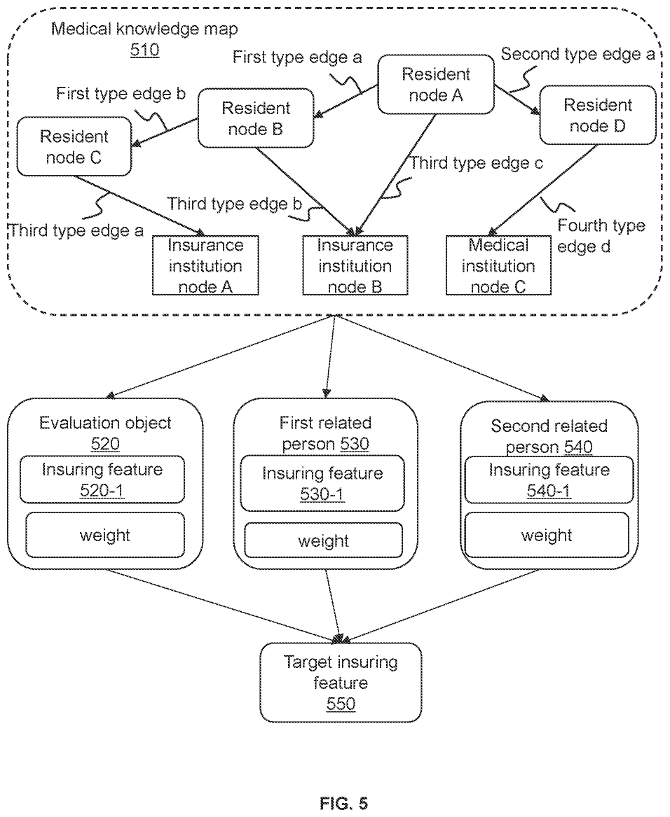

In some embodiments, the medical management platform 230 may be configured to further perform the following operations: obtaining a synergistic feature of the evaluation object based on the synergistic platform; and determining the evaluation value of the insurance risk based on the target medical feature, the target insuring feature, and the synergistic feature. For more details of the synergistic features of the evaluation objects and the related persons, please refer to FIG. 5 and related descriptions.

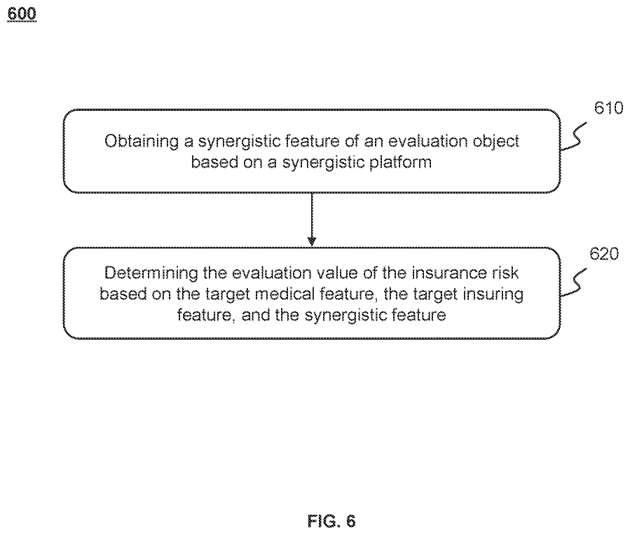

In some embodiments, the medical management platform 230 may be configured to further perform the following operations: through processing the target medical feature vector and the target insuring feature vector based on a risk evaluation model, determining the evaluation value of the insurance risk. For more details about the evaluation value of the insurance risk, please refer to FIG. 6 and the related descriptions.

It should be noted that the above description of the system and its components is only for the convenience of description, and is not intended to limit the scope of the present disclosure. It should be appreciated that for those skilled in the art, after understanding the principle of the system, each component may be arbitrarily combined, or may form sub-systems to connect with other components without departing from this principle. For example, each component may share one storage device, or each component may further have their own storage device. Such deformations are within the protection range of the present disclosure.

FIG. 3 is an exemplary flowchart illustrating a method for evaluating medical insurance data in a smart city based on the IoT according to some embodiments of the present disclosure. As shown in FIG. 3, a process 300 includes the following operations. In some embodiments, the process 300 may be performed by a medical management platform 230.

In 310, a risk query request of a user may be obtained based on a service platform, the risk query request may be used to evaluate an insurance risk of an evaluation object through a user platform.

In some embodiments, the user may include relevant operators of insurance companies, related people who have insured or intended to be insured.

The evaluation object may refer to a person whose insurance risk need to be evaluated. For example, when a user a intends to take out the critical illness insurance, the insurance risk of user a may be evaluated (or assessed), and the user a may be the evaluation object.

The insurance risk may refer to a risk that may lead to insurance claims after the evaluation object is insured. In some embodiments, the insurance risk may be a risk of failure to pay a subsequent premium caused by the poor economic state of the evaluation object.

The risk query request may refer to an operation instruction used to perform risk evaluation on the insurance risk of the evaluation object. In some embodiments, the risk query request may include the evaluation object and a type of insurance that has been insured or intended to be insured by the evaluation object. For example, the risk query request may be that: performing risk evaluation on the critical illness insurance taken by an evaluation object a. The evaluation object in the risk query request may be identified through identification information. For example, the risk query request may include the ID number and the name, etc. of the evaluation object.

In some embodiments, the user platform 210 may generate a risk query request based on the identification information and insurance type (or type of insurance) of the evaluation object entered by the user platform. In some embodiments, the medical management platform 230 may obtain the risk query request entered by the user platform 210 via the service platform 220. For example, a relevant operator of an insurance company may enter the risk query request in the user platform 210. After receiving the risk query request, the user platform 210 may send the risk query request to the service platform 220. The service platform 220 may analyze the risk query request and send it to the medical management platform 230 so that the medical management platform 230 may perform the method for evaluating medical insurance data in a smart city based on the IoT provided by the present disclosure according to the risk query request. More about the user platform 210, the service platform 220, and the medical management platform 230 may be found in FIG. 2 and related descriptions.

In 320, a related person of the evaluation object may be obtained based on a population information platform.

The population information platform may refer to a cloud platform or an external database that records the information related to residents (such as ID information, address information of the residents, etc.). For example, the population information platform may include a household registration database of the public security system.

In some embodiments, the medical management platform 230 may, in response to the risk query request, communicate with the population information platform, and obtain the related person of the evaluation object.

The related person may refer to a person who have relationship with the evaluation object. In some embodiments, the related person may be a person who has a kinship with the evaluation object. For example, the related person may be the wife, husband, parent, brother, or sister of the evaluation object. In some embodiments, the related person may be a person who has a certain relationship with the address, the disease record, the treatment record, or insuring record, etc., of the evaluation object. For example, the related person may be a person who has insured the same type of insurance as the evaluation object. For another example, the related person may be a person with the same residence address as the evaluation object.

In some embodiments, the medical management platform 230 may find out the persons who have kinship with the evaluation object or have a certain relationship with the evaluation object in the addresses, disease records, treatment records or insurance records through the population information platform, and determine these persons as the related persons of the evaluation object.

In some embodiments, the medical management platform 230 may retrieve (or call) relevant information of the evaluation object from the population information platform according to the identification information of the evaluation object, and determine the related person according to the relevant information of the evaluation object. For example, according to the address of the evaluation object, a person having the same address with the evaluation object may be taken as the related person of the evaluation object. In some embodiments, to ensure the privacy of the evaluation object and related person, the evaluation object and related person fed back by the population information platform may be characterized by identification information that does not expose private information (such as ID number).

In 330, the medical features and insuring features of the evaluation object and the related person may be obtained based on a medical information platform.

The medical information platform may refer to a cloud platform or an external database that records the relevant medical information of the residents. In some embodiments, the medical information platform may include information such as disease records, treatment records, insuring records (including insurance types and claims of commercial medical insurances purchased, etc.) of the residents.

A medical feature may be a feature describing the health status of an object (i.e., the evaluation object and related person). In some embodiments, the medical feature may include the number of treatments for the disease, the cost of treatments, etc. In some embodiments, the medical feature may include a first medical feature and a second medical feature. The first medical feature may be a medical feature related to genetic diseases (such as asthma, congenital heart disease, etc.). The second medical feature may be a medical feature related to diseases caused by unhealthy lifestyles (such as hypertension, diabetes, etc.). In some embodiments, the medical feature may further include those related to ordinary diseases (such as colds, fever, etc.). More on the first medical feature and the second medical feature may be found in FIG. 4 and the related descriptions.

An insuring feature may be a feature describing types and claims of a medical insurance purchased by the object (such as the evaluation object and the related person). In some embodiments, the insuring feature may include the insurance type, the number of times to take insurance, the total insurance premium, the number of times of claims, and the total amount of claims.

In some embodiments, the medical management platform 230 may send a data call request to the medical information platform to obtain the medical features and insuring features of the evaluation object and the related person. The data call request may include the identification information of the object (such as the ID of the object), the data type (such as, the medical feature or the insuring feature), etc. In some embodiments, the medical information platform may analyze the corresponding data call request, determine the ID of the evaluation object and the related person, and call the corresponding data according to the ID and send the data to the medical management platform 230.

In some embodiments, when obtaining the medical features and the insuring features of the evaluation object and the related person based on the medical information platform, the medical management platform 230 may obtain a medical knowledge map based on the medical information platform, and obtain the medical features and the insuring features of the evaluation object and the related person based on the medical knowledge map.

The medical knowledge map may refer to a semantic network built based on medical data and insurance data of the residents. The medical knowledge map may include node data and edge data.

The node data may include resident nodes and institution nodes (or institution nodes). One resident node corresponds to a resident, and a node attribute corresponding to the resident node may include age information and address information, etc. of the resident. One institutional node corresponds to an institution. The institution node may include a medical institution node and an insurance institution node, and a node attribute corresponding to the institution node may include the name of the institution, the address of the institution, etc.

The edge data may include types and attributes of the edges. The type of an edge (or type edge) between the resident nodes may include edges that indicate the existence of a first-generation immediate family relationship (hereinafter referred to as a first type edges), and edges that indicate the same address (hereinafter referred to as a second type edges). The types of the edges between the resident nodes and the institution nodes may include the edges indicating the existence of insurance relationships between the resident nodes and the corresponding insurance institutions (hereinafter referred to as a third type edge), and the edges indicating the existence of the treatment relationships between the resident nodes and the corresponding medical institutions (hereinafter referred to as the fourth type edge).

The first type edge may be used to indicate that there is a first-generation immediate family relationship between residents corresponding to resident nodes. The first-generation immediate family relationship may include father-son relationships, mother-daughter relationships, etc. What fist-generation immediate family relationship between two connected nodes is may be determined based on the attribute of the first type of edge connecting the two nodes.

The second type edge may be used to indicate that the resident nodes that the second type edge connected has the same residential address. For example, there may be a second type edge between the resident nodes corresponding to the residents living in the Lotus Community. The specific address information between two connected nodes may be determined based on the attribute of the second type edge connecting the two nodes.

The third type edge may be used to indicate the existence of an insurance relationship between the resident and the insurance institution to reflect that the resident has brought insurance in the insurance institution. In some embodiments, edge attribute of the third type edge may include the number of times for insuring, the total insurance premium, the number of times of claims, and the total amount of claims. In some embodiments, the edge attribute of the third type edge may be represented by a vector. For example, the edge attribute of the third type edge may be (a, b, c, d), wherein a denotes the number of times of insurance, b denotes the total insurance premium, c denotes the number of times of claims, and d denotes the total amount of claims.

The fourth type edge may be used to represent a treatment relationship between a resident and a medical institution, which reflects that the resident has been treated in the medical institution. In some embodiments, the edge attribute of the fourth type edge may include a treatment frequency vector and a treatment cost vector. In some embodiments, the treatment frequency vector and the treatment cost vector may be three-dimensional (3D) vectors, and the three dimensions may respectively be related to the first medical feature, the second medical feature, and a third medical feature. For example, there may be an edge between a resident a and a medical institution b, and the treatment frequency vector may be (0,0,5), the treatment cost vector may be (0,0,500). Then the vectors indicate that the resident a has been treated for 5 ordinary diseases in the medical institution b, and the cost is CNY 500.

In some embodiments, the medical management platform 230 may find corresponding resident nodes from the medical knowledge map according to the identification information of the evaluation object and the related person, and may determine the node and the edge related to the each of resident nodes. Then, the medical features and the insuring features of the evaluation object and the related person may be determined according to the node attributes (or the attribute of the node) and the edge attributes (or the attribute of the edge). In some embodiments, the insuring feature of the evaluation object may be determined according to the edge attribute of the third type edge between the resident node and the insurance institution node corresponding to the evaluation object. For example, the edge attribute of the third type edge may be (2,6000,1,20000), then the corresponding insuring feature may be determined as that the number of times of insurance is 2, the total insurance premium is CNY 6,000, the number of claims is 1, the total amount of claims is CNY 20000.

In some embodiments, the medical feature of the evaluation object may be determined according to the edge attribute of the fourth type edge between the resident node and the medical institution node corresponding to the evaluation object. The edge attribute of the fourth type edge may include the treatment frequency vector, and the treatment cost vector, etc. In some embodiments, the treatment frequency vector and the treatment cost vector may be three-dimensional (3D) vectors, and the three dimensions may respectively be related to the first medical feature, the second medical feature, and the third medical feature. For example, there may be an edge between resident a and medical institution b, the treatment frequency vector may be (0,0,5), the treatment cost vector may be (0,0,500). Then, the vectors indicate that resident a has been treated for 5 ordinary diseases in medical institution b, and the cost is CNY 500. In some embodiments, the treatment frequency vector and treatment cost vector of the edge attribute of the fourth type edge may be determined as the medical features of the evaluation object.

In some embodiments, the related person of the evaluation object may be determined according to the first type edge and the second type edge connected with the resident node corresponding to the evaluation object, and then the insuring feature and the medical feature of the related person may be respectively determined according to the third type edge and the fourth type edge connected with the resident node corresponding to the related person. Similarly, the way of determining the insuring feature and the medical feature of the related person may be the same as the way of determining the insuring feature and the medical feature of the evaluation object.

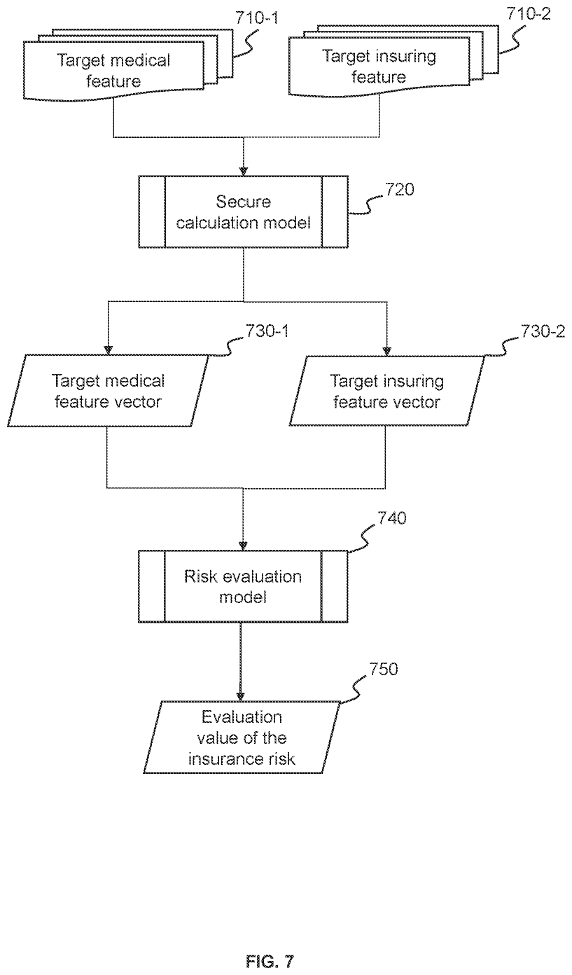

In some embodiments, considering that the medical feature and the insuring feature may involve the privacy information of the evaluation object, before the medical information platform sends the insuring features and the medical features of the evaluation object and the related person to the medical management platform, the insuring features and the medical features may be encrypted through a secure calculation model, and a medical feature vector and an insuring feature vector may be generated. The vectors may not reveal the privacy information of the evaluation object. More about the secure calculation model may be found in FIG. 7 and the related descriptions.

In 340, a target medical feature and a target insuring feature may be determined based on the medical features and the insuring features of the evaluation object and the related person.

The target medical feature may reflect the impact of the related person on the medical feature of the evaluation object. In some embodiments, the target medical feature may be a weighted result of the medical features of the related person and the evaluation object itself. Similarly, the target insuring feature may reflect the impact of related person on the insuring feature of the evaluation object. In some embodiments, the target insuring feature may be a weighted result of the insuring features of the related person and the evaluation object itself.

In some embodiments, the medical management platform 230 may perform weighting processing on the medical features of the evaluation object and the related person to determine the target medical feature. The medical management platform 230 may perform weighting processing on the insuring features of the evaluation object and the related person to determine the target insuring feature.

In some embodiments, the medical management platform 230 may assign different weights to the medical feature of the evaluation object and the medical feature of the related person, and then perform weighting processing to determine the target medical feature. For example, the weight of the medical feature of the evaluation object may be set to 0.8, the weight of the medical feature of the related person a may be 0.08, the weight of the medical feature of the related person b may be 0.1, and the weight of the medical feature of the related person c may be set to 0.02 to determine the target medical feature. Similarly, the medical management platform 230 may assign different weights to the insuring feature of the evaluation object and the insuring feature of the related person, and then perform weighting processing to determine the target insuring feature.

In some embodiments, the medical management platform 230 may assign different weights to the first medical feature of the evaluation object and the first medical feature of the related person, and then perform weighting processing to determine the weighted first medical feature. The medical management platform 230 may assign different weights to the second medical feature of the evaluation object and the second medical feature of the related person, and then perform weighting processing to determine the weighted second medical feature. Finally, the target medical feature may be determined based on the weighted first medical feature and the weighted second medical feature. Exemplarily, the number of times of treatments and the costs in the first medical feature of a first related person may be weighted into the first medical feature of the evaluation object; and the number of times of treatments and the costs in the second medical feature of a second related person may be weighted into the second medical feature of the evaluation object, then the target medical feature of the evaluation object may be obtained. More content about the weighted first medical feature and the weighted second medical feature may be found in FIG. 4 and the related descriptions.

In some embodiments, the weight may be related to a proximity of the resident node corresponding to the evaluation object to the resident node corresponding to the related person. The smaller the proximity is, the higher the weight is. The proximity may be related to the number of edges involved in the shortest path between the two nodes. A proximity of 1 means that the shortest path between two nodes is one edge. More content about proximity may be found in FIG. 4 and the related descriptions.

In some embodiments, the weights of the evaluation object and the related person may reflect the influence of the related person on the evaluation object. For example, a relative with a larger proximity may have a larger difference between the genes of the relative and the evaluation object, and the weight of the relative may be lower. When relatives with smaller proximities suffer from gene-related diseases, the weights of the relatives may be higher, and the probability of the evaluating object with the disease-related disease may be higher. In some embodiments, the weight of the evaluation object may be greater than the weight of the related person. In some embodiments, the weight can further be preset in advance. For example, when the proximity is 1, the weight may be 0.3 and when the proximity is 2, the weight may be 0.2.

In some embodiments, the medical management platform 230 may determine the first related person and the second related person based on the medical knowledge map, as well as determining the first medical feature of the first related person and the second medical feature of the second related person. The medical management platform 230 may obtain the weighted first medical feature by performing weighting processing on the first medical feature of the first related person and the first medical feature of the evaluation object, and obtain the weighted second medical feature by performing weighting processing on the second medical feature of the second related person and the second medical feature of the evaluation object. Finally, the medical management platform 230 may determine the target medical feature based on the weighted first medical features and the second medical features. For more content on determining the target medical feature, see FIG. 4 and the related descriptions.

In some embodiments, the medical management platform 230 may determine the first related person and the second related person based on the medical knowledge map, and determine the insuring features of the first related person and the second related person. Then, the medical management platform 230 may perform weighting processing on the insuring feature of the first related person, the insuring feature of the second related person, and the insuring feature of the evaluation object to determine the target insuring feature. More content about determining the target insuring feature, may be found in FIG. 5 and the related descriptions.

In 350, an evaluation value of the insurance risk may be determined based on the target medical feature and the target insuring feature.

The evaluation value may be used to quantify the insurance risk. A larger evaluation value indicates a higher insurance risk for the evaluation object and a higher probability for the insurance company to compensate against the evaluation object. In some embodiments, the performance of the evaluation value can be determined according to actual needs. For example, the evaluation value may be in percentage or may be in risk levels (such as risk levels 1-3, the higher the value is, the higher the risk level will be).

In some embodiments, the medical management platform 230 may perform processing on the target medical feature and the target insuring feature based on the secure calculation model, and determine a target medical feature vector and a target insuring feature vector. Then, the medical management platform 230 may perform processing on the target medical feature vector and the target insuring feature vector based on a risk evaluation model. In some embodiments, the secure calculation model and the risk evaluation model may be obtained through joint training. More content about the risk evaluation model may be found in FIG. 7 and the related descriptions.

Considering that the target medical feature and the target insuring feature may involve the privacy information of the object, before the medical information platform obtains the target medical features and the target insuring features of the evaluation object and the related person, the target insuring features and the target medical features may be encrypted through the secure calculation model by the medical management platform 230, and the target medical feature vectors and the target insuring medical feature vectors may be generated. The vectors may prevent the revealing of the privacy information of the object. More content about the secure calculation model and the risk evaluation model may be found in FIG. 7 and the related descriptions.

In 360, the evaluation value may be fed back to the user through the user platform and based on the service platform.

In some embodiments, when the medical management platform 230 determines the evaluation value, the evaluation value may be sent to the user platform through the service platform, and the evaluation value may be fed back to the user through the user platform.

In some embodiments, the user may perform continuous process based on the evaluation value obtained. In some embodiments, the user may adjust the insurance costs of the evaluation object based on the evaluation value, or refuse the insurance request of the evaluation object. For example, the user may increase premium of the insurance project for a higher evaluation value or refuse the evaluation object to participate in the insurance project. In some embodiments, the user may arrange more detailed examinations on the evaluation object based on the evaluation value. For example, the insurance company may further arrange the evaluation object to conduct medical examinations in their designated medical institution to obtain more detailed medical data.

Based on the method for evaluating medical insurance data in a smart city based on the IoT provided by some embodiments of the present disclosure, the evaluation value of the insurance risk of the evaluation object may be provided for the user (such as the insurance assessor) without revealing the privacy information of the evaluation object. Then, the insurance strategy of the evaluation object may be adjusted based on the evaluation value of the insurance risk (such as adjusting the insured premium of the evaluation object, or refuse the insurance request of the evaluation object). The insurance risk of the evaluation object may be determined based on the relevant conditions of the related person of the evaluation object (such as insuring condition and illness condition), so as to reasonably arrange the insurance price and project for the evaluation object, thereby reducing the economic cost of medical insurance.

It should be noted that the description of the above flow 300 is only for examples and descriptions, and should not limit the scope of the present disclosure. For those skilled in the art, under the guidance of the present disclosure, various amendments and changes may be made on the flow 300. However, these amendments and changes are still within the scope of the present disclosure. For example, the above operation 330 and operation 340 may be performed by the medical information platform or other relevant processing devices.

FIG. 4 is an exemplary schematic diagram illustrating the obtaining of a target medical feature according to some embodiments of the present disclosure.

As shown in FIG. 4, node data of a medical knowledge map 410 may include a resident node A, a resident node B, a resident node C, a resident node D, a medical institution node A, a medical institution node B, an insurance institution node C, etc. Edge data of the medical knowledge map 410 may include a first type edge a, a first type edge b, a second type edge a, a fourth type edge a, a fourth type edge b, a fourth type edge c, and a third type edge d, etc. More content of the resident node, the medical institution node, the first type edge, the second type edge, the third type edge, and the fourth type edge, may be found in FIG. 3 and the related descriptions.

In some embodiments, a first related person 430 and a second related person 440 of an evaluation object 420, as well as a first medical feature 430-1 of the first related person 430 and a second medical feature 430-2 of the second related person 440 may be determined based on the medical knowledge map 410.

The first related person may be a person corresponding to a resident node which has a first type edge with a resident node corresponding to an evaluation object, that is, the first related person may be a person who has an immediate family relationship with the evaluation object. As shown in FIG. 4, there may be a first type edge a between the resident node A and the resident node B. When the resident corresponding to the resident node A is the evaluation object, and the resident corresponding to the resident node B may be the first related person of the evaluation object.

The second related person may be the person corresponding to the resident node which has a second type edge with the resident node corresponding to the evaluation object, that is, the second related person may be a person who has the same address with the evaluation object. As shown in FIG. 4, there may be a second type edge a between the resident node A and the resident node D. When the resident corresponding to the resident node A is the evaluation object, the resident corresponding to the resident node D may be the second related person of the evaluation object.

In some embodiments, the first related person 430 of the evaluation object 420 can be determined according to a preset proximity condition. The proximity may be related to the number of edges involved in the shortest path between two nodes. A zero-proximity indicates that there is no edge between the two nodes. A one-proximity indicates that the shortest path between the two nodes involves one edge. A two-proximity indicates that the shortest path between the two nodes involves two edges. For example, resident node A may be connected with resident node B through the first type edge a, and the resident node B may be connected with the resident node C through the first type edge b. That is, there may be two first type edges between the resident node A and the resident node C. The preset proximity condition may be a condition that the proximity between two nodes needs to meet. For example, a preset proximity value may not be greater than 2.

It should be noted that in the embodiment of determining the first related person, when the proximity is greater than or equal to 2, the edges between the two nodes must be of the same type. For example, when the proximity is 2, the two edges between the two nodes may both be the first type edges or the second type edges, but they cannot be a first type edge and a second type edge.

In some embodiments, when the edge type is the first type, the resident corresponding to the resident node, wherein the proximity between the resident node and the resident node corresponding to the evaluation object meets the preset proximity condition, may be determined as the first related person of the evaluation object. Exemplarily, the preset proximity condition is that the proximity is 2. When the resident node corresponding to the evaluation object 420 in the medical knowledge map is resident node A, the residents corresponding to the resident nodes having one or two first type edges with the resident node A may be determined as the first related persons 430 of the evaluation object 420, that is, the resident corresponding to the resident node B, who has a first type edge a with the resident node A, and the resident corresponding to the resident node C, who has a first type edge b with the resident node B, may be determined as the first related persons 430.

In some embodiments, the resident corresponding to the resident node who has second type edge with the resident node corresponding to the evaluation object may be determined as the second related person of the evaluation object. Exemplarily, when the resident node corresponding to the evaluation object 420 in the medical knowledge map is resident node A, the resident corresponding to the resident node having a second type edge with the resident node A may be determined as the second related person 440 of the evaluation object 420, that is, the resident corresponding to the resident node D who has a second type edge a with the resident node A may be determined as the second related person 440.

In some embodiments, after determining the first related person 430 and the second related person 440, the medical management platform 230 may determine a first medical feature 430-1 of the first related person 430 and a second medical feature 440-1 of the second related person 440 according to edge attributes of the medical knowledge map 410.

In some embodiments, according to the edge attributes of fourth type edges between the medical institution nodes and the resident nodes respectively corresponding to the first related person 430 and the second related person 440, the first medical feature 430-1 of the first related person 430 and the second medical feature 440-1 of the second related person 440 may be determined. For example, the relevant data of gene-related diseases in the medical features of the first related person 430 may be determined as the first medical feature 430-1 of the first related person 430. For another example, the relevant data of the disease caused by unhealthy lifestyles in the medical features of the second related person 440 may be determined as the second medical feature 440-1 of the first related person 440. More content about obtaining medical features may be found in operation 330 and related descriptions, which will not be repeated here.

In some embodiments, the first medical feature 420-1 and the second medical feature 420-2 of the evaluation object 420 may be directly determined according to the medical features of the evaluation object 420. For example, the relevant data of gene-related diseases in the medical features of the evaluation object 420 may be determined as the first medical feature 420-1 of the evaluation object 420, the relevant data of the disease caused by unhealthy lifestyles in the medical features of the evaluation object 420 may be determined as the second medical feature 420-2 of the evaluation object 420. More content about obtaining medical features of the evaluation object may be found in operation 330 and the related descriptions, which will not be repeated here.

In some embodiments, the medical management platform 230 may perform weighting process on the first medical feature 430-1 of the first related person 430 and the first medical feature 420-1 of the evaluation object 420 to obtain a weighted first medical feature 450. Further, the medical management platform 230 may perform weighting process on the second medical feature 440-1 of the second related person 440 and the second medical feature 420-2 of the evaluation object 420 to obtain a weighted second medical feature 460. Finally, the medical management platform 230 may determine a target medical feature 470 based on the weighted first medical feature 450 and the weighted second medical feature 460.

Exemplarily, when the resident corresponding to the resident node A is the evaluation object 420, the medical feature of the evaluation 420 in the medical institution B may be determined according to the edge attribute of the fourth type edge c between the medical institution node B and the resident node A. That is, a treatment frequency vector and a treatment cost vector may respectively be (0,0,10) and (0,0,5000), and the corresponding weight of the evaluation object 420 may be 0.5. According to the edge attribute of the fourth type edge b between the resident node B and the medical institution node B, the treatment frequency vector and the treatment cost vector corresponding to the first related person 430 may respectively be determined as (1,0,0), and (10000,0,0), and the corresponding weight of the first related person 430 corresponding to the resident node B may be 0.2. According to the edge attribute of the fourth type edge d between the resident node D and the medical institution node B, the treatment frequency vector and the treatment cost vector corresponding to the second related person 440 may respectively be determined as (0,2,0), and (0,20000,0), and the corresponding weight of the second related person 440 corresponding to the resident node D may be 0.3. The treatment frequency vector and the treatment cost vector between the second related person 440 corresponding to the resident node D and the medical institution B may respectively be (0,0,10) and (0,0,5000), and the weight may be 0.5. Then, a weighted medical feature of the evaluation object may be determined, that is, a weighted treatment frequency vector and a weighted treatment cost vector may respectively be (0.2,0.6,5) and (2000,6000,2500). Finally, a weighted first medical feature 450 may be (0.2,20000), wherein 0.2 denotes the treatment frequency of the first medical feature, and 2000 denotes the treatment cost of the first medical feature. A weighted second medical feature 460 may be (0.6,6000), wherein 0.6 denotes the treatment frequency of the second medical feature and 6000 denotes the treatment cost of the second medical feature.

Based on the target medical feature determination provided by some embodiments of the present disclosure, by obtaining a weighted result of the medical features of the evaluation object and the related person, the impacts of health status of the family, family genetic diseases, and living lifestyle of the cohabitant on the insurance risks of the evaluation object may be fully considered. In this way, the risk evaluation on the target object mat be more accurate.

FIG. 5 is an exemplary schematic diagram illustrating the obtaining of a target insuring feature according to some embodiments of the present disclosure.

As shown in FIG. 5, node data of a medical knowledge map 510 may include a resident node A, a resident node B, a resident node C, a resident node D, a medical institution node A, a medical institution node B, an insurance institution node C, etc. Edge data of the medical knowledge map 510 may include a first type edge a, a first type edge b, a second type edge a, a third type edge a, a third type edge b, a third type edge c, a fourth type edge d, etc. More content of the resident nodes, the medical institution nodes, the first type edges, the second type edges, the third type edges, and the fourth type edges may be found in FIG. 3 and the related descriptions.

As shown in FIG. 5, there may be the first type edge a between the resident node A and the resident node B. When the resident corresponds to the resident node A is the evaluation object, the resident corresponding to the resident node B may be a first related person of the evaluation object. There may be the second type edge a between the resident node A and the resident node D. When the resident corresponds to the resident node A is the evaluation object, the resident corresponding to the resident node D may be a second related person of the evaluation object.

In some embodiments, when the edge type is the first type, the resident corresponding to the resident node, wherein the proximity between the resident node and the resident node corresponding to the evaluation object meets a preset proximity condition, may be determined as the first related person of the evaluation object. In some embodiments, the resident corresponding to the resident node having the second type edge with the resident node corresponding to the evaluation object may be determined as the second related person of the evaluation object. More about determining the first related person and the second related person may be found in FIG. 4 and related descriptions.

In some embodiments, after determining the first related person 530 and the second related person 540, the medical management platform 230 may determine an insuring feature 530-1 of the first related person 530 and an insuring feature 540-1 of the second related person 540 based on the edge attributes of the medical knowledge map 510.

In some embodiments, according to the edge attribute of the third type edge between the resident node and the insurance institution node respectively corresponds to the first related person 530 and the second related person 540, the insuring feature 530-1 of the first related person 530 and the insuring feature 540-1 of the second related person 540 may be determined.

In some embodiments, the insuring feature 520-1 and the insuring feature 520-2 of the evaluation object 520 may be directly determined according to the insuring feature of the evaluation object 520. The method of determining the insuring feature may be similar to that of determining the first medical feature and the second medical feature. More content may be found in FIG. 4 and the related descriptions.

In some embodiments, the medical management platform 230 may perform weighting process on the insuring feature 530-1 of the first related person 530, the insuring feature 540-1 of the second related 540 and the insured feature 520-1 of the evaluation object 520 to obtain a target insuring feature 550. The determination method of the target insuring feature 550 may be similar to that of the target medical feature 470. More content may be found in FIG. 4 and the related descriptions.

Based on the target insuring feature determination provided by some embodiments of the present disclosure, by obtaining a weighted result of the insuring feature of the evaluation object and the insuring feature of the related person, the historically-purchased commercial medical insurance and claims of the evaluation object can be fully considered, and the risk evaluation can be more accurately performed on the target object.

FIG. 6 is an exemplary flowchart illustrating the determination of an evaluation value of an insurance risk based on a synergistic feature according to some embodiments of the present disclosure. In some embodiments, a flow 600 may be performed by a medical management platform 230. As shown in FIG. 6, the flow 600 may include following operations.

In 610, a synergistic feature of an evaluation object may be obtained based on a synergistic platform.

The synergistic feature may refer to other feature data that may be used to determine a value of evaluating an insurance risk of an evaluation object (i.e., the evaluation value of the insurance risk of an evaluation object). In some embodiments, the synergistic feature may include a financial synergistic feature reflecting asset situation of the evaluation object, a social relief synergistic feature reflecting application of social relief or receiving social relief by the evaluation object, and a vehicle insurance claim synergistic feature reflecting a vehicle insurance claim situation of the evaluation object, etc.

The synergistic platform refers to one or more cloud platforms or external databases recorded with relevant synergistic information of the residents. In some embodiments, the synergistic platform may include a financial platform, a social relief platform, and a traffic management platform. The financial platform may refer to a cloud platform or an external database that records the financial information of the residents (such as deposits, loans, credit card usage, etc.). For example, the financial platform may include the resident database of the central bank. The social relief platform may refer to a cloud platform or an external database that records social relief information of the residents (such as application for relief funds, receiving relief funds, application for relief materials, receiving relief materials, etc.). For example, the social relief platform may include an aid distribution database of a civil affairs system. The traffic management platform may refer to a cloud platform or an external database that records the vehicle insurance information from the residents (such as vehicle insurance condition, vehicle insurance claim situation, etc.). For example, the traffic management platform may include a vehicle database of a vehicle management system.

In some embodiments, the medical management platform 230 may obtain the synergistic features of the evaluation object and the related person based on the synergistic platform. For example, the medical management platform 230 may call the corresponding synergistic features from the synergy platform according to the identification information (such as ID) of the evaluation object. In some embodiments, considering that the synergistic features may involve privacy information of the evaluation object and the related person, the synergistic platform may encrypt the synergistic information through a secure calculation model before sending the synergistic features to the medical management platform, and the synergistic features may be generated without exposing the privacy information of the object. More content about the secure calculation model may be found in FIG. 7 and the related descriptions.

In some embodiments, the medical management platform 230 may obtain the financial features of the evaluation object and the related person based on a financial knowledge map.

The financial feature may refer to a feature describing the financial information of an object (such as the evaluation object and the related person). In some embodiments, the financial feature may include the total number of times of overdue credit card, the total amount of overdue credit card.

The financial knowledge map may refer to a semantic network built based on the residents' financial information. The financial knowledge map may include node data and edge data.

The node data of the financial knowledge map may include resident nodes and financial institution nodes. The resident node corresponds to a resident, and the node attribute corresponding to the resident node may include age information and address information of the resident. The financial institution node corresponds to a financial institution. The financial institution may refer to an institution that provides financial services (such as banks). The node attribute corresponding to the financial institution node may include address information of the financial institution, etc.

The edge data of the financial knowledge map may include a type of an edge and an attribute of the edge. The type of the edge between resident nodes may include the edge with the same address (i.e., a second type edge) of residents, and the type of the edge between the resident node and the financial institution node may include the edge which has financial businesses with the corresponding financial institution (hereinafter referred to as a fifth type edge). The fifth type edge may be used to indicate that the credit card of the resident which is applied in the corresponding financial institution is overdue. In some embodiments, the edge attribute of the fifth type edge may include the number of times of overdue credit card, the total amount of overdue credit card. In some embodiments, when obtaining the financial feature, the person corresponding to the resident node having the second type edge with the resident node corresponding to the evaluation object may be regarded as the related person.

In some embodiments, the financial feature of the evaluation object may be determined by the financial knowledge map based on the edge attribute of the fifth type edge. For example, the total number of times of overdue credit card and the total amount of overdue credit card of the evaluation object may be determined as the financial feature of evaluation object by calculating a sum of the number of times of overdue credit card and the amount of overdue credit card of the evaluation object in each financial institution. In some embodiments, the related person of the evaluation object may be determined according to the resident node corresponding to the evaluation object, and the financial feature of the related person may then be determined. The determination mode of the financial feature of the related person may be similar to that of the financial feature of the evaluation object.

In some embodiments, weighting processing may be performed on the financial features of the evaluation object and the related person to obtain financial synergistic information based on the financial knowledge map. For example, weighting processing may be performed on the total number of times of credit card overdue and the total amount of credit card overdue of the evaluation object and the related person who lives together with the evaluation object, so as to determine the financial synergistic information of the evaluation object (that is, the weighted results of the total number of times of credit card overdue, and the weighted results of the total amount of credit card overdue).

In some embodiments, before the financial synergistic information is sent to the medical management platform 230 by the financial platform, the financial synergistic information may be processed by the secure calculation model to determine the financial synergistic feature. It should be understood that through the secure calculation model, the financial synergistic information may be encrypted, and a financial synergistic feature vector (referred to as a financial synergistic feature) may be generated. The financial synergistic feature vector may not disclose the privacy information of the object. More content about the secure calculation model may be found in FIG. 7 and the related descriptions.

Based on the financial platform shown in some embodiments of the present disclosure as a synergistic platform, the synergistic feature may reflect the comprehensive financial ability of the evaluation object, so as to further consider the evaluation object's ability to pay the premium when determining the evaluation value of the insurance risk, and improve the accuracy of the evaluation value of the insurance risk.

In some embodiments, the medical management platform 230 may determine the social relief features of the evaluation object and the related person through a social relief knowledge map.

The social relief features may be features describing the social relief information of the object (such as the evaluation object and the related person). In some embodiments, the social relief features may include a frequency or times of picking free food, a total value of the food received, a frequency or times of the relief applications, a total amount of the relief applications, etc., of the object.

The social relief knowledge map may refer to a semantic network built based on residents' social relief information, including node data and edge data.

The node data of the social relief knowledge map may include resident nodes and relief institution nodes. The resident node corresponds to a resident, and the node attribute of the resident node may include age information and address information of the resident, etc. The relief institution node corresponds to a relief institution. The relief institution may refer to an institution that provides social relief services (such as free food pickup points). The node attribute of the relief institution node may include address information of the relief institution, etc.

The edge data of the social relief knowledge map may include a type and an attribute of an edge. The type of the edge between resident nodes include an edge indicate that the residents have the same address (i.e., the second type edge), and the type of the edge between the resident node and the social relief institution node may include an edge indicating that a resident has received free food in the social relief institution (hereinafter referred to as the sixth type edge) and an edge indicating that a resident has applied for relief in the social relief institution (hereinafter referred to as the seventh type edge).

The sixth type edge may be used to indicate that the resident has received free food in the social relief institution. In some embodiments, the edge attribute of the sixth type edge may include the frequency (or number of times or times) of receiving (picking) free food, and the total value of the free food. For example, resident A has been gone to a free food pickup point B and a free food pickup point C to receive free food, then there may be a sixth type edge between A and B and between A and C. In some embodiments, a resident node has a plurality of sixth type edges and the corresponding number of times of picking free food exceeds a certain threshold, indicating that the resident has received free food from the various institutions, and the feature may be taken as the social relief feature of the resident node.

The seventh type edge may be used to indicate that the resident has applied for relief in the social relief institution. In some embodiments, the edge attribute of the seventh type edge may include a frequency (or number of times) of relief applications and a total amount of the relief applications, etc.

In some embodiments, when obtaining the social relief feature, the person corresponding to the resident node having the second type edge with the resident node corresponding to the evaluation object may be regarded as a related person.

In some embodiments, the social relief feature of the evaluation object may be determined by the social relief knowledge map according to the edge attributes of the sixth type edge and the seventh type edge connected to the resident node corresponding to the evaluation object. For example, the total number of times or total frequency of picking the free food, the total value of food received, the total number of times or total frequency of relief application and the total amount of relief application of the evaluation object may be determined by calculating the sum of the number of times or frequency of picking the free food, the value of food received, the number of times or frequency of relief application and the amount of relief application of the evaluation object in each social relief institution, and may be taken as the social relief feature of the evaluation object. In some embodiments, the related person of the evaluation object may be determined according to the resident node corresponding to the evaluation object, and then the social relief feature of the related person may be determined. The determination mode may be similar to that of determining the social relief feature of the evaluation object.

In some embodiments, weighting processing may be performed on the social relief features of the evaluation object and the related person to obtain social relief synergistic information based on the social relief knowledge map. For example, weighting processing may be performed on the total number of times or total frequency of picking free food, the total value of food received, the total number of times or total frequency of relief applications and the total amount of relief applications of the evaluation object and the related person lives with the evaluation object to obtain the social relief synergistic information of the evaluation object (that is, the weighted result of number of times or total frequency of picking free food, the weighted result of the value of food received, the weighted result of the number of times or total frequency of relief applications, and the weighted result of the amount of relief applications).

In some embodiments, before the social relief synergistic information is sent to the medical management platform 230 by the social relief platform, the social relief synergistic information may be processed by the secure calculation model to determine the social relief synergistic feature. It should be understood that through the secure calculation model, the social relief synergistic information may be encrypted, and a social relief synergistic feature vector (referred to as a social relief synergistic feature) may be generated. The social relief synergistic feature vector may not reveal the privacy information of the object. More content about the secure calculation model may be found in FIG. 7 and the related descriptions.

Based on the social relief platform shown in some embodiments of the present disclosure as a synergistic platform, the synergistic feature may reflect the comprehensive social relief situation of the evaluation object, so as to further consider the evaluation object's social relief situation when determining the evaluation value of the insurance risk, and improve the accuracy of the evaluation value of the insurance risk.

In some embodiments, the medical management platform 230 may obtain the vehicle insurance claim feature of the evaluation object through the traffic management platform.

The vehicle insurance claim feature may refer to the feature of vehicle insurance claim information of the object (such as evaluation object and related person). In some embodiments, the vehicle insurance claim feature may include the number of times of the vehicle insurance claim of the evaluation object, the average maintenance amount, the average claim amount, etc.

In some embodiments, the traffic management platform may call the vehicle insurance claim information based on identification information (such as ID) of the evaluation object. In some embodiments, before the traffic management platform sends the vehicle insurance claim information to the medical management platform 230, the vehicle insurance claim information may be processed by the secure calculation model to determine the vehicle insurance claim feature. It should be understood that through the secure calculation model, the vehicle insurance claim information may be encrypted, and a vehicle insurance claim feature vector (referred to as a vehicle insurance claim feature) may be generated. The vehicle insurance claim feature vector may not reveal the privacy information of the object. More content about the secure calculation model may be found in FIG. 7 and the related descriptions.

Based on the traffic management platform shown in some embodiments of the present disclosure as a synergistic platform, the vehicle insurance claim feature as a synergistic feature, when determining the evaluation value of the insurance risk, the insurance applications and claims of other insurance types of the evaluation object may be further considered, thereby improving the accuracy of the evaluation value of the insurance risk.

In 620, based on the target medical feature, the target insuring feature, and the synergistic feature, the evaluation value of the insurance risk may be determined.

In some embodiments, the synergistic feature may be used as a certain factor affecting the evaluation value of the insurance risk. For example, the premium payment ability of the evaluation object may be estimated according to the synergistic feature, e.g., financial synergistic feature and social relief synergistic feature, and then the evaluation value of the insurance risk may be adjusted. For another example, the evaluation value of the insurance risk may be adjusted according to an estimated probability of claims of the evaluation object after vehicle accidents.

In some embodiments, the medical management platform 230 may perform processing on the target medical feature, the target insuring feature and the synergistic feature based on a risk evaluation model to determine the evaluation value of the insurance risk. More content about the risk evaluation model may be found in FIG. 7 and the related descriptions.

Based on the synergistic feature provided by the embodiments of the present disclosure, the relevant information of the evaluation object can be further reflected, thereby improving the accuracy of the evaluation value of the insurance risk.

FIG. 7 is an exemplary schematic diagram illustrating the determination of an evaluation value of the insurance risk according to some embodiments of the present disclosure.

In some embodiments, a medical management platform 230 may perform processing on a target medical feature 701-1 and a target insuring feature 710-2 through a secure calculation model 720 to determine a target medical feature vector 730-1 and a target insuring feature vector 730-2. More content on the target medical feature and the target insuring feature may be found in FIG. 3-FIG. 5 and the related descriptions.

The secure calculation model 720 may be used to extract the target medical feature vector 730-1 and the target insuring feature vector 730-2. In some embodiments, the secure calculation model 720 may be a machine learning model, for example, recurrent neural network (RNN). In some embodiments, the input of the secure calculation model 720 may be the target medical feature 710-1 and the target insuring feature 710-2, the output may be the target medical feature vector 730-1 and the target insuring feature vector 730-2.

It is worth noting that the target medical feature 710-1 and the target insuring feature 710-2 may contain a large amount of privacy data of evaluation objects. Considering the data security problem of a medical information platform, after receiving the calling request of the medical management platform, the medical information platform may perform multi-party secure calculation processing on the target medical feature 710-1 and the target insuring feature 710-2. The target medical feature vector 730-1 and the target insuring feature vector 730-2 after processing may reflect the target medical feature of the evaluation object through encrypted data, which does not involve the specific data of the evaluation object.

The multi-party secure calculation may ensure that the information entered by members of different parts participating in the calculation is not exposed in the absence of a trusted third party, and accurate calculation results can be obtained. In some embodiments, the medical information platform, the synergistic platform, and the medical management platform may encrypt their respective data through multi-party secure calculation. For example, the medical information platform may encrypt the target medical feature and the target insuring feature through the secure calculation model. The synergistic platform may encrypt financial synergistic information, social relief synergistic information and vehicle insurance claim information through the secure calculation model. The encrypted data information may be processed through a risk evaluation model, and the evaluation value of the insurance risk may be calculated.

In some embodiments, the medical management platform 230 may process the target medical feature vector 730-1 and the target insuring feature vector 730-2 based on the risk evaluation model 740 to determine the evaluation value 750 of the insurance risk.

The risk evaluation model 740 may be used to determine the evaluation value of the insurance risk of the evaluation object. In some embodiments, the risk evaluation model 740 may be a machine learning model, for example, a deep neural network (DNN). In some embodiments, the input of the risk evaluation model 740 may include the target medical feature vector 730-1 and the target insuring feature vector 730-2, and the output may be the evaluation value 750 of the insurance risk.

In some embodiments, the input of the risk evaluation model 740 may further include synergistic features, such as financial synergistic features, social relief synergistic features, vehicle insurance claim features, etc. More content about the synergistic features may be found in FIG. 5 and the related descriptions.

In some embodiments, the input of the risk evaluation model 740 may be the output of the secure calculation model 720, that is, the target medical feature vector 730-1, the target insuring feature vector 730-2, and the synergistic feature. The output may be the evaluation value 750 of the insurance risk.

In some embodiments, the secure calculation model 720 may be obtained by joint training with the risk evaluation model 740. For example, the training samples may be input to the secure calculation model, and the output of the secure calculation model may be used as the input of the risk evaluation model. Based on the output of the risk evaluation model, a loss function may be constructed. The parameters of the secure calculation model and the risk evaluation model may be simultaneously iterated, until a preset condition is satisfied, and the training ends.

In some embodiments, a training sample may include a sample target medical feature of a sample evaluation object, a sample target insuring feature, and a sample synergistic feature. The training sample may be obtained based on a historical evaluation data. The historical evaluation data may be the historical data of evaluation on the insurance risks of the sample evaluation object, and the label may be the evaluation value of the insurance risk of the sample evaluation object. For example, a user A may be determined as the sample evaluation object before Feb. 2, 2020. A network cloud platform may obtain historical target medical feature, historical target insuring feature and historical synergistic features of the user and its related person before Feb. 2, 2020, and take them as the training sample of the secure calculation model. Further, the historical evaluation value of the insurance risk of the user (e.g., the higher the total claim amount, the higher the assessed value) may be determined according to the claims after Feb. 2, 2020 (such as the total amount of claims, the number of times of claims, etc.). The historical evaluation value may be taken as the corresponding label of the training sample to perform model training. For another example, the historical evaluation value of the user A may be evaluated manually by the staffs of the insurance institutions according to factors like the occupation and family income etc. of the user A. Exemplarily, for an ordinary employee of a company, the actual evaluation value may be a normal value (e.g., evaluation values between 40-60 points may be normal values). For people with high positions and live in richer residential areas (usually with good health status), the actual evaluation value may be lower (e.g., evaluation values between 0-40 points may be lower values). For people with severe diseases or people who are engaged in special types of works (such as miners, as they may suffer from occupational disease risks), their actual evaluation value may be higher (e.g., evaluation values between 60-100 points may be higher values).

In some embodiments, when the secure calculation model 720 may be jointly trained with the risk evaluation model 740, the training may be carried out synergistically through multi-platforms, that is, at least one platform of the network cloud platform 150 may cooperate with the medical management platform 230 to perform training. For example, as one of the plurality of parties, the medical information platform and the financial platform may jointly train the secure calculation model 720 and the risk evaluation model 740. During training, the medical information platform may first determine the sample evaluation object and a corresponding sample related person. For example, the information of the sample evaluation object and the sample related person may be sent to a plurality of platforms of the network cloud platform 150 to obtain the relevant sample feature data of the sample evaluation object and the sample related person (e.g., sample financial feature, sample social relief feature, sample vehicle insurance claim feature, etc.). Then, the relevant sample feature data may be input to the secure calculation model 720, and the output of the secure calculation model 720 may be used as the input of the risk evaluation model 740. Finally, a loss function may be constructed based on the output of the risk evaluation model 740, and the parameters of the secure calculation model 720 and the risk evaluation model 740 are simultaneously iterated based on the loss function, until a preset condition is satisfied, and the training ends. After the training ends, one or more platforms of the network cloud platform may obtain the corresponding secure calculation model 720, and the medical information platform may obtain the corresponding risk evaluation model 740.

In some embodiments, the risk evaluation model may not be jointly trained with the secure calculation model as well. For example, the risk evaluation model may be trained according to the sample target medical feature vector and sample target insuring feature vector obtained in advance. For another example, the risk evaluation model may be trained based on a trained secure calculation model.

By determining the evaluation value of the insurance risk provided by the embodiments of the present disclosure, the target medical feature and the target insuring feature can be processed through the risk evaluation model, thereby accurately and quickly determining the evaluation value of the insurance risk. In some embodiments, by adding the security calculation model and the risk evaluation model to jointly determine the evaluation value of the insurance risk, the privacy of the evaluation object can be well protected.

The basic concept has been described above. Obviously, for the those skilled in the art, the above detailed disclosure is only used as an example, and it does not constitute a limitation of the present disclosure. Although it is not clearly stated here, those skilled in the art may make modifications, improvements, and amendments to the present disclosure. The modifications, improvements, and amendments are proposed in the present disclosure, so the modifications, improvements, and amendments of this type still belong to the spirit and scope of the embodiments of the present disclosure.

At the same time, the present disclosure uses a specific word to describe the embodiments of the present disclosure. For example, “one embodiment”, “one implementation example”, and/or “some embodiments” means a feature, structure or features related to at least one embodiment related to the present disclosure. Therefore, it should be emphasized and noticed that in the present disclosure, “one implementation example” or “one embodiment” or “an alternative embodiment” that are mentioned in different positions in the present disclosure does not necessarily mean the same embodiment. In addition, some features, structures, or characteristics of one or more embodiments in the present disclosure may be properly combined.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

Finally, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

We claim:

1. A method for evaluating medical insurance data in a smart city based on the Internet of Things (IoT), which is performed by a medical management platform, comprising:
obtaining, based on a service platform, a risk query request of a user through a user platform, wherein the risk query request is used to evaluate an insurance risk of an evaluation object;
obtaining, based on a population information platform, a related person of the evaluation object;
obtaining, based on a medical information platform, medical features and insuring features of the evaluation object and the related person;
determining, based on the medical features and insuring features of the evaluation object and the related person, a target medical feature and a target insuring feature;
determining, based on the target medical feature and the target insuring feature, an evaluation value of the insurance risk; and
feeding back, based on a service platform, the evaluation value to the user through the user platform.

2. The method of claim 1, wherein the obtaining, based on a medical information platform, medical features and insuring features of the evaluation object and the related person comprises:
obtaining, based on the medical information platform, a medical knowledge map; and
obtaining, based on the medical knowledge map, the medical features and insuring features of the evaluation object and the related person.

3. The method of claim 1, wherein the determining, based on the medical features and insuring features of the evaluation object and the related person, a target medical feature and a target insuring feature comprises:
determining the target medical feature by performing weighting processing on the medical features of the evaluation object and the related person; and
determining the target insuring feature by performing weighting processing on the insuring features of the evaluation object and the related person.

4. The method of claim 3, wherein each of the medical feature of the evaluation object and the medical feature of the related person at least include a first medical feature and a second medical feature, and
the determining the target medical feature by performing weighting processing on the medical features of the evaluation object and the related person comprises:
determining, based on the medical knowledge map, a first related person and a second related person and determining a first medical feature of the first related person and a second medical feature of the second related person;
obtaining a weighted first medical feature by performing weighting processing on the first medical feature of the first related person and the first medical feature of the evaluation object;
obtaining a weighted second medical feature by performing weighting processing on the second medical feature of the second related person and the second medical feature of the evaluation object; and
determining, based on the weighted first medical feature and the weighted second medical feature, the target medical feature.

5. The method of claim 4, wherein the determining the target insuring feature by performing weighting processing on the insuring features of the evaluation object and the related person comprises:
determining, based on the medical knowledge map, an insuring feature of the first related person and an insuring feature of the second related person; and
determining the target insuring feature by performing weighting processing on the insuring feature of the first related person, the insuring feature of the second related person, and the insuring feature of the evaluation object.

6. The method of claim 1, wherein the determining, based on the target medical feature and the target insuring feature, an evaluation value of the insurance risk comprises:
determining a target medical feature vector and a target insuring feature vector by processing the target medical feature and the target insuring feature based on a secure calculation model; and
determining the evaluation value of the insurance risk by processing the target medical feature vector and the target insuring feature vector based on a risk evaluation model.

7. The method of claim 6, wherein the secure calculation model and the risk evaluation model are obtained through joint training.

8. The method of claim 1, further comprising:
obtaining, based on a synergistic platform, a synergistic feature of the evaluation object; and
determining, based on the target medical feature, the target insuring feature, and the synergistic feature, the evaluation value of the insurance risk.

9. The method of claim 8, wherein the synergistic platform at least includes a financial platform, a social relief platform and a traffic management platform, and the synergistic feature at least includes a financial synergistic feature, a social relief synergistic feature, and a vehicle insurance claim synergistic feature.

10. A system for evaluating medical insurance data in a smart city based on the Internet of Things, comprising a user platform, a service platform and a medical management platform, wherein the medical management platform is configured to perform the following operations comprising:
obtaining, based on a service platform, a risk query request of a user through a user platform, wherein the risk query request is used to evaluate an insurance risk of an evaluation object;
obtaining, based on a population information platform, a related person of the evaluation object;
obtaining, based on a medical information platform, medical features and insuring features of the evaluation object and the related person;
determining, based on the medical features and insuring features of the evaluation object and the related person, a target medical feature and a target insuring feature;
determining, based on the target medical feature and the target insuring feature, an evaluation value of the insurance risk; and
feeding back, based on a service platform, the evaluation value to the user through the user platform.

11. The system of claim 10, wherein the medical management platform is configured to further perform the following operations comprising:
obtaining, based on the medical information platform, a medical knowledge map; and
obtaining, based on the medical knowledge map, the medical features and insuring features of the evaluation object and the related person.

12. The system of claim 10, wherein the medical management platform is configured to further perform the following operations comprising:
determining the target medical feature by performing weighting processing on the medical features of the evaluation object and the related person; and
determining the target insuring feature by performing weighting processing on the insuring features of the evaluation object and the insuring features of the related person.

13. The system of claim 12, wherein each of the medical feature of the evaluation object and the medical feature of the related person at least include a first medical feature and a second medical feature, and
the medical management platform is configured to further perform the following operations comprising:
determining, based on the medical knowledge map, a first related person and a second related person and determining the first medical feature of the first related person and a second medical feature of the second related person;
obtaining a weighted first medical feature by performing weighting processing on the first medical feature of the first related person and the first medical feature of the evaluation object;
obtaining a weighted second medical feature by performing weighting processing on the second medical feature of the second related person and the second medical feature of the evaluation object; and
determining, based on the weighted first medical feature and the weighted second medical feature, a target medical feature.

14. The system of claim 13, wherein the medical management platform is configured to further perform the following operations comprising:
determining, based on the medical knowledge map, an insuring feature of the first related person and an insuring feature of the second related person; and
determining the target insuring feature by performing weighting processing on the insuring feature of the first related person, the insuring feature of the second related person, and the insuring feature of the evaluation object.

15. The system of claim 10, wherein the medical management platform is configured to further perform the following operations comprising:
determining a target medical feature vector and a target insuring feature vector by processing the target medical feature and the target insuring feature based on a secure calculation model; and
determining the evaluation value of the insurance risk by processing the target medical feature vector and the target insuring feature vector based on a risk evaluation model.

16. The system of claim 15, wherein the secure calculation model and the risk evaluation model are obtained through joint training.

17. The system of claim 10, wherein the medical management platform is configured to further perform the following operations comprising:
obtaining, based on a synergistic platform, a synergistic feature of the evaluation object; and
determining, based on the target medical feature, the target insuring feature, and the synergistic feature, the evaluation value of the insurance risk.

18. The system according to claim 17, wherein the synergistic platform at least includes a financial platform, a social relief platform and a traffic management platform, and the synergistic feature at least includes a financial synergistic feature, a social relief synergistic feature, and a vehicle insurance claim synergistic feature.

19. A computer-readable storage medium storing computer instructions, wherein when reading the computer instructions in the storage medium, a computer implements the method for evaluating medical insurance data in a smart city based on the IoT according to claim 1.
